(19) United States
(12) Patent Application Publication
(10) Pub. No.: US 2003/0135240 A1
(43) Pub. Date: Jul. 17, 2003

(54) Magnetic induced current therapy

(76) Inventor: Joseph Patrick Dimino, Phoenix, AZ (US)

Correspondence Address: Joseph Patrick Dimino, 11th Place, 7020 N., Phoenix, AZ, 85020-5311, US

(21) Appl. No.: 09/981,355

(22) Filed: Jan. 14, 2002

(51) Int. Cl. A61N 1/00

(52) U.S. Cl. 607/2

(57) ABSTRACT

This invention is for the elimination of viral and bacterial pathogens by using electric current induced by a strong, moving magnetic field. This same invention can also be used to sanitize biological transplants, blood, and medical utensils. I have found that currents of 50-300 micro amperes, when produced by a moving magnetic field are sufficient enough to damage most micro organisms rendering them incapable of reproducing themselves. I must emphasize, the key here is not magnetism, nor electric shock from an electrical source—The success of this treatment is based on the penetrating quality of magnetism that allows a moving field to produce current evenly throughout a hosts body. While the small current is sufficient enough to kill germs it will not harm the host.

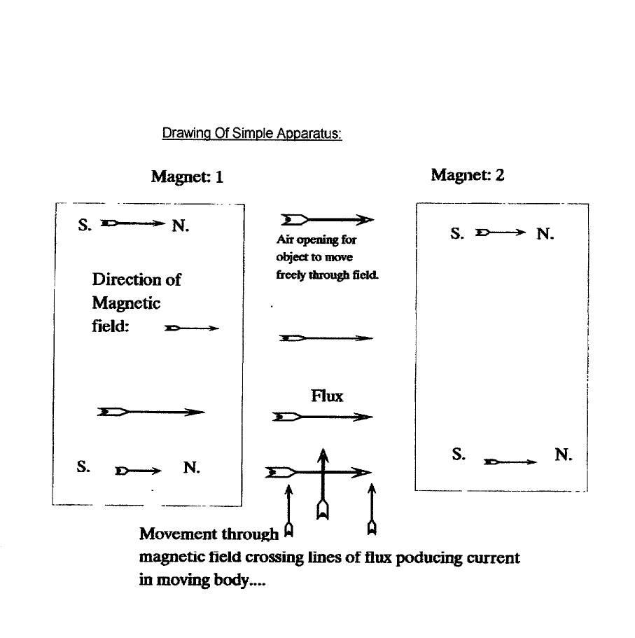

This invention is for the elimination of viral and bacterial pathogens by using electric current induced by a strong, moving magnetic field. This same invention can also be used to sanitize biological transplants, blood, and medical untensils. I have found that currents of 50-300 micro amperes, when produced by a moving magnetic field are sufficient enough to damage most micro organisms rendering them incapable of reproducing themselves. While electric shock in the past has been tested on micro organisms to see the effects, my treatment is different in the respect that the current must be produced by a moving magnetic field if it is to be destructive to a pathogen. Current at levels safe for human tissues by itself is ineffective, because the medium or tissues harboring the pathogen (as does the exterior body of the pathogen itself) protects their sensitive interior from the shocking effect of electric current. Similar short comings apply to treatments of pathogens using a magnetic field without moving the field to produce current: A strong field by itself will not destroy the pathogen and in some cases will do the opposite, cause harmful organisms to proliferate. I must emphasize, the key here is not magnetism, nor electric shock from an electrical source—The success of this treatment is based on the penetrating quality of magnetism that allows a moving field to produce current evenly and throughout a hosts body and the body of any infecting micro organism. The same effect occurs when a patient is moved through a stationary magnetic field. The motion of the patient through the field immediately produces current throughout his body and the body of any harmful organism he may be harboring. While I believe the current used in this treatment is small enough not to harm a patient (50-300 micro amps), it is more than sufficient to seriously damage the vital interiors of harmful pathogens. There is nothing in a human body that can redirect or obstruct a magnetic field. A magnetic field will penetrate the body as far as the field extends. When the field is then moved, it produces current throughout the entire body and thus throughout any micro organism infecting its human host. Of course there is considerable testing to be completed. Right now I am confident that this treatment would be ideal and safe for eliminating infections from human extremities. I have scientific reason to believe that slight amounts of low voltage current when applied to human nerve tissue and heart tissue (the entire body at one time), for short duration (just seconds for each treatment) would be well tolerated. Much more research needs to be done . . . though I have strong preliminary scientific theory leading me to believe that the entire human body subjected to low voltage/current, electric shock throughout, for short duration, would simply shut down (mild form of shock), and immediately start up when the current was discontinued leaving no noticeably distress or discomfort.

This Invention covers any apparatus producing a magnetic field whose movement through or movement of manifests a current in a living host or micro organism for the purpose of destroying or weakening harmful or disease producing pathogens . . . . This same invention can also be used to sanitize biological transplants, blood, and medical utensils.

1. This invention claims that if an electric current of 50-300 micro ampere is produced by a moving magnetic field, any virus and bacteria passed through the current will be destroyed.
